(19) United States
(12) Patent Application Publication
(10) Pub. No.: US 2011/0212165 A1
(43) Pub. Date: Sep. 1, 2011

(54) PHARMACEUTICAL COMPOSITIONS OF THE ISOLATED D-ENANTIOMER OF THE QUINAZOLINONE DERIVATIVE HALOFUGINONE

(76) Inventors: Mordechai Erez, Tel Aviv (IL); Karin Halevi, Kadima (IL)

(21) Appl. No.: 13/102,730

(22) Filed: May 6, 2011

Related U.S. Application Data
(63) Continuation of application No. 11/816,839, filed on Aug. 22, 2007.
(60) Provisional application No. 60/655,413, filed on Feb. 23, 2005.

(51) Int. Cl. A61K 9/127; A61K 31/517; A61P 37/06; A61P 17/14; A61P 17/06; A61P 17/08; A61P 17/10; A61P 1/16; A61P 9/10; A61P 35/00; C07D 401/06

(52) U.S. Cl. 424/450; 514/266.22; 544/287

(57) ABSTRACT

The present invention relates to pharmaceutical compositions comprising as an active ingredient an isolated, chirally pure D-enantiomer of the quinazolinone derivative halofuginone having increased therapeutic activity and decreased side effects compared to the corresponding racemic mixtures, the composition being substantially free of the L-enantiomer and useful in the treatment of diseases and disorders associated with fibrotic conditions or cell proliferation.

### FIELD OF THE INVENTION

The present invention relates to pharmaceutical compositions comprising as an active ingredient an isolated, chirally pure D-enantiomer of the quinazolinone derivative halofuginone, having increased therapeutic activity and decreased side effects compared to the corresponding racemic mixtures, the composition being substantially free of the L-enantiomer and useful in the treatment of diseases and disorders associated with fibrotic conditions or cell proliferation.

### BACKGROUND OF THE INVENTION

Quinazolinone Derivatives

Quinazolinone derivatives for treating coccidiosis were disclosed and claimed in U.S. Pat. No. 3,320,124. Halofuginone, otherwise known as 7-bromo-6-chloro-3-[3-(3-hydroxy-2-piperidinyl)-2-oxopropyl]-4(3H)-quinazolinone (one of the quinazolinone derivatives), was first described and claimed in said patent to American Cyanamid, and was the preferred compound taught by said patent and the one commercialized from among the derivatives described and claimed therein.

Subsequently, U.S. Pat. Nos. 4,824,847; 4,855,299; 4,861,758 and 5,215,993 relate to the coccidiocidal properties of halofuginone. U.S. Pat. No. 4,340,596 teaches the use of lactate salts of quinazolinone derivatives for the treatment of theileria in cattle. Halofuginone hydrobromide is marketed for veterinary use as Stenorol®, and is used as an additive in chicken feed. The chemical characterization, toxicology and pharmacokinetics of halofuginone are well documented (NADA document #130-951 (SBA), 1985). Halofuginone hydrobromide is marketed for veterinary use as Stenorol®, and is used as an additive in chicken feed.

U.S. Pat. No. 5,449,678 teaches quinazolinone derivatives useful for the treatment of fibrotic conditions such as scleroderma and graft versus host disease (GVHD). The '678 disclosure provides compositions of a fibrosis inhibitor comprising a therapeutically effective amount of a pharmaceutically active compound having general formula I:

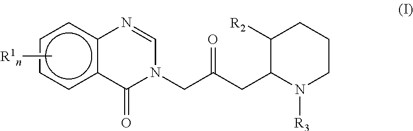

wherein: n=1-2; and

R₁ which may be the same or different at each occurrence is a member of the group consisting of hydrogen, halogen, nitro, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl.

Of this group of compounds, halofuginone was found to be particularly effective.

U.S. Pat. Nos. 6,028,075 and 6,090,814 disclose the use of pharmaceutical compositions comprising quinazolinones, specifically halofuginone for treating malignancies and preventing neovascularization, respectively. International Application WO 03/059355 teaches that these compounds are also useful in improving the effectiveness of antitumor treatments and in the prevention of damage induced by radiation therapy.

Progressive fibroproliferative diseases such as liver cirrhosis, pulmonary and kidney fibrosis, scleroderma and a variety of other serious diseases, exhibit excessive production of connective tissues, which results in the destruction of normal tissue architecture and function. The fibrotic reaction is thought to involve the stimulative response of tissue cells resulting in increased proliferation as well as extracellular matrix (ECM) deposition. Collagen was found to be a major ECM molecule synthesized in fibrotic lesions. Halofuginone has been shown to be effective in inhibiting collagen type I gene expression at low concentrations thus providing broad therapeutic utility of halofuginone as an antifibrotic drug.

U.S. Pat. No. 5,891,879 discloses quinazolinone derivatives effective in treating restenosis. Both fibrosis and restenosis are conditions associated with excessive collagen deposition, which can be inhibited by halofuginone. Restenosis is characterized by smooth muscle cell proliferation and extracellular matrix accumulation within the lumen of affected blood vessels in response to vascular injury (Choi et al., Arch. Surg., 130:257-261, 1995). The phenotypic alteration of proliferative smooth muscle cells has been shown to be regulated by collagen type I, which can be blocked by halofuginone (Choi et al., ibid 1995; U.S. Pat. No. 5,449,678). U.S. Pat. No. 6,159488 discloses the use of halofuginone as a coating for intracoronary stents.

In addition to the fibrotic diseases with excess collagen deposition, normal wound healing involves the formation of connective tissue that consist largely of collagen fibrils. Although moderate degrees of fibrous tissue are beneficial in wound repair, excess fibrous material often accumulates and impairs the normal function of the affected tissue. Such excessive accumulation of collagen becomes an important event in scarring of the skin after burns or traumatic injury, in hypertrophic scars and in keloids. PCT publication WO 01/17531—teaches halofuginone as effective in wound healing.

The pathophysiological response to tissue trauma may vary in different tissue types, but often results in the formation of scars or other types of connective tissues which lack the functionality of the original organ tissue, so that the repair of tissue trauma does not lead to a complete restoration of organ capacity and function. EP patent No. 1109559 discloses that quinazolinone derivatives, particularly halofuginone, are effective in preventing the pathogenic aspects of tissue trauma while preserving normal tissue repair mechanism.

Halofuginone was shown to be an effective agent for use in the treatment of various tissue disorders including hepatic cirrhosis (U.S. Pat. No. 6,562,829), renal fibrosis (International (PCT) Application WO 02/094178), pulmonary fibrosis (International Application WO 98/43642) wounds (International Application WO 01/17531) and adhesions (U.S. Pat. No. 5,852,024). U.S. Pat. No. 6,211,188 discloses the use of halofuginone for the treatment of skin disorders, including psoriasis, hypertrophic scars and keloids,

International Application WO 03/070153 discloses stabilized compositions of halofuginone and methods for decreasing the rate of isomerization of the halofuginone trans isomer to the cis isomer by addition of an acidic component to the pharmaceutical composition.

Stereoisomers

Stereoisomers are compounds made up of the same atoms bonded by the same sequence of bonds but having different three-dimensional structures, which are not interchangeable. Optically active compounds, which have one chiral atom or more, exist as two or more isomers, called enantiomers. Enantiomers are minor images of one another and have identical physical properties, except for the fact that they rotate the plane of polarized light in opposite directions, (+) clockwise for the dextro isomer, and (−) counterclockwise for the levo isomer. Likewise, they have identical chemical properties except when interacting with stereospecific compounds. When the rates at which each enantiomer reacts or interacts with another chiral compound are sufficiently different, a clear divergence in activity is observed, and many compounds that are biologically active have inactive enantiomers.

Halofuginone is currently synthesized and available commercially as Stenorol® which is a racemic mixture. U.S. Pat. No. 4,252,947 teaches a process for the synthesis of an active dextrorotary enantiomer of halofuginone for the treatment of coccidiosis. The dextrorotary compound was shown to have three times the coccidiostatic activity of the racemic compound, however was found to be about two times more toxic. That patent does not disclose the purity of the obtained dextrorotary enantiomer obtained by selective synthesis. The '947 patent does not disclose pharmaceutical grade compositions of dextrorotary halofuginone suitable for human use, which requires sufficiently high purity level of the active ingredient as well as the excipient(s). While the dextrorotary enantiomer of halofuginone was demonstrated to be the more active enantiomer when used as an anti-parasitic food supplement for chicken, a priori it was not possible to predict which enantiomer exerts the therapeutic effects of halofuginone on fibrotic and proliferative diseases in mammals, particularly as the purity of the examined D-enantiomer was not described.

Halofuginone has yet to be approved for any human use. However, in preclinical testing and in early human clinical testing halofuginone hydrobromide has been shown to be highly effective in the treatment of a wide variety of diseases and disorders, yet the side effects, including nausea and emesis, renders it intolerable to some subjects, particularly at higher doses.

It would be advantageous to have active formulations of halofuginone for use as a treatment for fibrosis and other diseases, while averting the side effects associated with the racemic compositions. Pharmaceutical compositions comprising an isolated, substantially pure enantiomer of halofuginone, which retains the beneficial biological activity while having reduced side effects associated with the racemic mixture would thus be highly desirable.

### SUMMARY OF THE INVENTION

The present invention provides enantiomerically pure quinazolinone derivatives, particularly halofuginone, for use as an active ingredient in pharmaceutical compositions for clinical applications. The present invention is based in part on the unexpected discovery that a pharmaceutical composition comprising the substantially purified D-enantiomer of halofuginone is useful for the treatment of a fibrotic condition as well as for the treatment of a disease or disorder associated with deleterious cell proliferation, including neoplastic diseases.

In the present invention, the D and L enantiomers of halofuginone were purified to have enantiomeric purity of at least 95%, preferably 98%, more preferably 99% or more. As disclosed herein for the first time, the purified D enantiomer of halofuginone is twofold more active in inhibiting cell proliferation and collagen synthesis than the racemic mixture, while the purified L-enantiomer is virtually inactive. In addition, it was unexpectedly discovered that some of the adverse side effects associated with pharmaceutical compositions comprising the racemic mixture of halofuginone were reduced in subjects treated with pharmaceutical compositions comprising the chirally pure D enantiomer of halofuginone.

Pharmaceutical compositions comprising the isolated biologically active enantiomer of halofuginone, having high chiral purity and substantially devoid of the opposite enantiomer, are highly desirable in the treatment of a variety of disorders receptive to treatment with halofuginone and derivatives thereof, where treatment with racemic halofuginone induces adverse side effects including nausea and vomiting in human subjects treated with the racemate. The pharmaceutical compositions of the present invention comprise the D enantiomer having at least 95% chiral purity. Thus, the present invention provides pharmaceutical grade compositions comprising isolated, chirally pure D-enantiomer of a quinazolinone derivative substantially devoid of the L-enantiomer, having the beneficial biological activity of the racemic mixture and decreased side effects, in different final dosage forms, and methods for use thereof.

According to one aspect the present invention provides a pharmaceutical composition for the treatment of diseases and disorders associated with fibrotic conditions or cell proliferation, the composition comprising as an active ingredient an isolated quinazolinone derivative having the general formula (Ia):

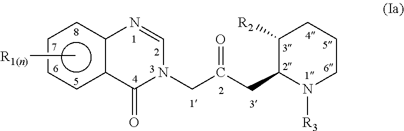

having the (2″R,3″S)trans configuration and being essentially free of the (2″S,3″R)trans enantiomer, wherein:

n=1-2,

R₁ is at each occurrence independently selected from the group consisting of hydrogen, halogen, nitro, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof, and a pharmaceutically acceptable carrier or diluent, the quinazolinone derivative being at least 95% chirally pure (2″R,3″S)trans enantiomer.

As used herein, “D-enantiomer” refers to the (2″R,3″S)trans configuration of quinazolinone of Formula Ia while the “L-enantiomer” refers to the (2″S,3″R)trans configuration.

According to certain embodiments, the composition comprises at least 98% chirally pure (2″R,3″S)trans enantiomer, preferably at least 99% or more (2″R,3″S)trans enantiomer.

The active compound is preferably D-enantiomer of halofuginone, a compound of formula (Ia) wherein n=2; R₁═Br and Cl; R₂═HO and R₃═H; a derivative thereof or a pharmaceutically acceptable salt of halofuginone.

According to certain currently preferred embodiments the present invention provides pharmaceutical compositions comprising the isolated D-enantiomer of halofuginone, essentially free of the L-enantiomer, the halofuginone being at least 95% chirally pure, preferably 98% chirally pure, more preferably at least 99% or more chirally pure D-enantiomer of halofuginone.

According to certain embodiments the pharmaceutical composition comprising the chirally pure D-enantiomer of a quinazolinone derivative exhibits reduced side effects compared to the side effects obtained upon administering a pharmaceutical composition comprising a racemic mixture of the quinazolinone derivative.

In certain embodiments, the reduced side effects include at least one reduced adverse effects selected from nausea, vomiting, salivation, diarrhea, apathy, low sperm count, vision problems, hypertension, hypotension, hypothermia and deviation from normal hematological counts.

In certain embodiments the pH of the composition is in a range of about 3.5 to about 8.5. According to one currently preferred embodiment, the pH of the composition is below 7.0, preferably below 6.5, more preferably below 6.0, most preferably below 5.5.

In certain embodiments the compound of formula Ia is formulated for oral, topical and/or parenteral administration. According to one embodiment, the pharmaceutical composition is formulated for oral administration, in liquid or solid forms. Liquid forms may be aqueous or non-aqueous. Currently preferred embodiments of the present invention relate to a solid pharmaceutical composition selected from the group consisting of tablets, capsules, sachets, powders, granules and lozenges.

According to certain currently preferred embodiments, the quinazolinone derivative is halofuginone, and the concentration of the isolated chirally pure D-enantiomer of halofuginone in the composition is in the range of from about 0.0001% to about 30% (w/w). In other embodiments the concentration of the isolated D-enantiomer of halofuginone in the composition is in the range of from about 0.001% to about 10%.

In some embodiments the pharmaceutical compositions of the present invention are formulated for topical use, the formulation selected from the group consisting of cream, ointment, lotion, gel, suspension, aqueous or cosolvent solutions, salve, liposomes and any other pharmaceutically acceptable carrier suitable for administration of the drug topically. Currently preferred topical formulations are selected from the group consisting of emulsions, non-washable (water-in-oil) creams or washable (oil-in-water) creams, lotions, salves, gels and the like.

Additional embodiments provide a pharmaceutical composition comprising the compound of formula Ia formulated for parenteral use. The pharmaceutical compositions for parenteral administration are preferably selected from the group consisting of sterile solutions ready for injection, sterile suspensions ready for injection, sterile dry soluble lyophilized powders ready for reconstitution by combination with a vehicle just prior to use, sterile emulsions, microemulsions, dispersions, liposomal delivery systems, lipid complexes such as with cholesterol derivatives and phospholipids.

Pharmaceutical compositions for parenteral administration are formulated in forms suitable for intravenous injections, intravenous infusion, intradermal, intralesional, intramuscular, and subcutaneous injections or depots; or they may be administered parenterally by means other than injection, for example, laparascopically, or intravesicularly.

According to certain embodiments the pharmaceutical compositions having as an active ingredient a compound according to general formula Ia are useful in the treatment of fibrotic conditions and proliferative diseases or disorders, which are responsive to treatment with quinazolinone compounds such as halofuginone. Relevant indications include skin diseases including psoriasis, keloid, hypertrophic scar, acne, seborrhea and alopecia. Preferably, the skin disorder is selected from the group consisting of psoriasis, keloid and hypertrophic scar. Other indications include fibrotic disease such as scleroderma, graft versus host disease (GVHD), hepatic cirrhosis, pulmonary fibrosis, and renal fibrosis. Proliferative indications include restenosis and malignant and non-malignant tumors.

According to another aspect, the present invention provides a method of treating a subject in need thereof, the method comprising administering to the subject a pharmaceutical composition comprising a therapeutically effective amount of an isolated quinazolinone derivative having the general formula (Ia)

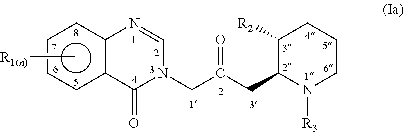

having the (2″R,3″S)trans configuration and being essentially free of the (2″S,3″R)trans enantiomer, wherein:

n=1-2,

R₁ is at each occurrence independently selected from the group consisting of hydrogen, halogen, nitro, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof, and a pharmaceutically acceptable carrier or diluent, the composition comprising at least 95% chirally pure (2″R,3″S)trans enantiomer of the quinazolinone derivative.

According to one embodiment, the isolated quinazolinone derivative of formula Ia is halofuginone, a derivative thereof or a pharmaceutically acceptable slat of halofuginone.

According to another embodiment, the subject in need is a human subject.

These and additional benefits and features of the invention could be better understood by those skilled in the art with reference to the following detailed description taken in conjunction with the figures and non-limiting examples.

### BRIEF DESCRIPTION OF THE FIGURES

The invention is herein described by way of example only, with reference to the accompanying drawings, wherein:

FIG. 1 shows the chemical structure of the D- and L-enantiomers of halofuginone.

FIG. 2 shows the effect of the D- and L-enantiomers (enantiomer 1 and 2, respectively) and the racemic mixture in inhibiting cell proliferation of Human aortic smooth muscle cells (hAoSMC) (FIG. 2A); Human Umbilical vein endothelial cells (HUVEC) (FIG. 2B); Human skin fibroblasts (Detroit 551) (FIG. 2C); Human bladder carcinoma (5637) (FIG. 2D); Human fibrosarcoma (HT108) (FIG. 2E); and human breast carcinoma (MDA-MB-435S) (FIG. 2F).

FIG. 3 shows the level of suppression of collagen I expression by the D- and L-enantiomers and the racemic mixture. (−)AA: cells which were not induced by ascorbic acid. NH₄TFA: induced cells without halofuginone and with the appropriate amount of the salt NH₄TFA.

### DETAILED DESCRIPTION OF THE INVENTION

The present invention relates to pharmaceutical compositions for the treatment of fibrotic and proliferative diseases and disorders comprising as an active ingredient an isolated, chirally pure D-enantiomer of a quinazolinone derivative, preferably halofuginone.

### Definitions

As used herein, the term “lower alkyl” refers to a straight- or branched-chain alkyl group of C₁ to C₆, for example, methyl, ethyl, propyl, isopropyl, butyl, isobutyl, sec-butyl, tert-butyl, pentyl, isopentyl, hexyl, isohexyl, and the like. The term “alkenyl” refers to a group having at least one carbon-to-carbon double bond.

The terms “alkoxy” and “alkenoxy” denotes —OR, wherein R is alkyl or alkenyl, respectively.

As used herein, the term “halogen” refers to fluorine, chlorine, bromine or iodine.

As used herein, “D-enantiomer” refers to (2″R,3″S)trans configuration of a quinazolinone derivative of formula Ia while the “L-enantiomer” refers to the (2″S,3″R)trans

As use herein, the terms “essentially free of the L-enantiomer” or “essentially free of the (2″S,3″R)trans enantiomer” refers to a quinazolinone compound that has less than about 5% L-enantiomer, preferably less than about 2% L-enantiomer, more preferably less than 1% L-enantiomer.

The terms “enantiomerically pure”, “enantiomeric purity”, “chiral purity” and “chirally pure” are used alternatively to reflect the fact that one enantiomer, generally the D-enantiomer [(2″R,3″S)trans when referring to compound of formula (Ia)], is found in the composition in greater proportion in relation to its mirror image. The proportion between two enantiomers is expressed by the absolute proportion of the D-enantiomer, which is at least 95% preferably 98% and more preferably 99% or more.

According to one aspect the present invention provides a pharmaceutical composition for the treatment of diseases and disorders associated with fibrotic conditions or cell proliferation, comprising as an active ingredient an isolated quinazolinone derivative having the general formula (Ia):

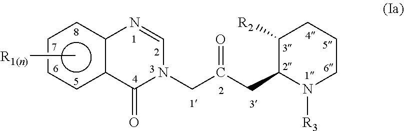

having the (2″R,3″S)trans configuration and being essentially free of the (2″S,3″R)trans enantiomer, wherein:

n=1-2,

R₁ is at each occurrence independently selected from the group consisting of hydrogen, halogen, nitro, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof, further comprising a pharmaceutically acceptable carrier or diluent, said composition comprising at least 95% chirally pure (2″R,3″S)trans enantiomer.

According to one currently preferred embodiment the active compound is halofuginone, or a pharmaceutically acceptable salt of halofuginone.

### Enantiomers of Halofuginone

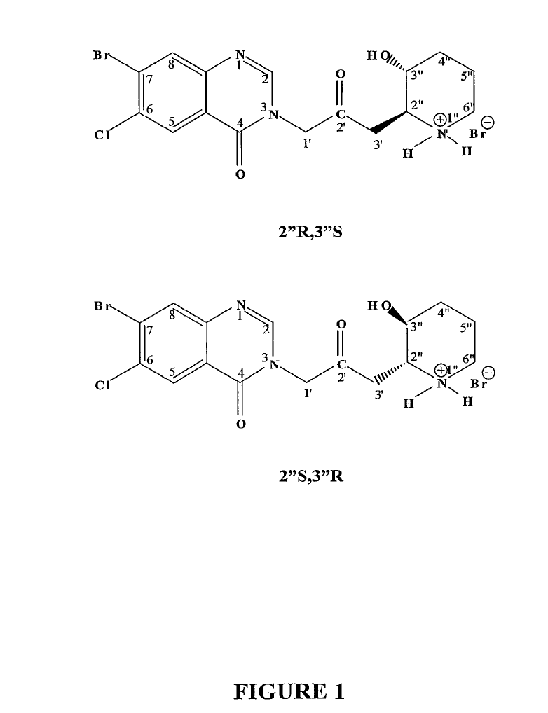

As shown in FIG. 1, halofuginone is an alkaloid containing a quinazolin-4-one moiety that is connected to a piperidinic ring by a ketonic bridge. The piperidinic ring is substituted on position 3″ with a hydroxyl group.

There are two chiral atoms in the molecule at positions 2″ and 3″ of the piperidinic ring, whereas halofuginone is synthesized as racemate at position 3″. The configuration at carbon 2″ interconverts spontaneously gradually in solutions with alkaline pH so that the configuration of the substituents interconvert between cis and trans as depicted in the following scheme.

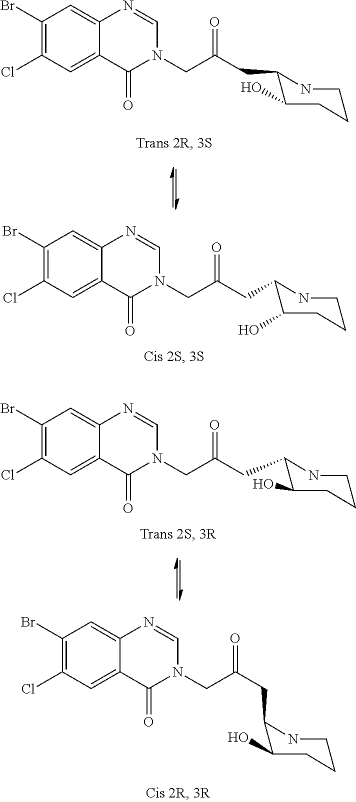

PCT Publication WO 03/070153, the content of which is incorporated herein by its entirety by reference, discloses that the trans isomer is the pharmaceutically active isomer, and that the rate of isomerization of the trans isomer to the cis isomer is reduced by adding an acidic component to the pharmaceutical composition and creating an acidic environment for the drug. The present invention now discloses that the chirally pure (2″R,3″S)trans enantiomer is the enantiomer having the anti fibrotic and anti proliferative biological activity; Thus, according to certain currently preferred embodiment, the pharmaceutical compositions of the present invention have a pH of below 7.0, as to stabilize the trans configuration of the D-enantiomer.

The enantiomeric purity of the active ingredient can be determined by types of tests known in the art. Typically, the chiral purity of halofuginone enantiomers according to the present invention is determined by analytical chiral HPLC.

According to certain embodiments, the halofuginone used in the pharmaceutical compositions of the present invention is characterized by at least 95% chiral purity, preferably by at least 98%, more preferably by at least 99% chiral purity.

The present invention demonstrates that the isolated D-enantiomer of halofuginone is the active enantiomer in inhibiting mammalian cell proliferation and collagen synthesis. Unexpectedly, in some subjects the isolated D-enantiomer of halofuginone demonstrates reduced undesirable side effects often seen upon administration of the racemic mixture. Thus, according to certain embodiments, the compositions and methods of the invention provide for decreasing the side effects in a subject associated with the administration of a racemic mixture of halofuginone.

Although the specific quinazolinone derivative “halofuginone” is referred to throughout the specification, it is understood that other quinazolinone derivatives according to formula I(a) may be used in its place.

Pharmaceutical Compositions

In certain preferred embodiment the present invention provides a pharmaceutical composition for topical, parenteral or oral administration. The use of the D-enantiomer in the preparation of pharmaceutical compositions, in indications responsive to halofuginone is desirable due to increased activity and decreased side effect concomitant with the retention of high activity.

According to certain currently preferred embodiments the concentration of the isolated D-enantiomer of halofuginone in the composition is in the range of 0.0001-30% (w/w), preferably in the range of 0.001 to 10% (w/w).

### Indications

By virtue of the anti-fibrotic and anti-proliferative properties of halofuginone it will be recognized that the compositions according to the present invention will be useful for treating indications having fibrotic or proliferative mechanism involved in their etiology or pathogenesis. The pharmaceutical compositions of the present invention, comprising the isolated chirally pure D-enantiomer of halofuginone are particularly useful in treating a subject in need thereof who is sensitive to the side effects induced by the racemic mixture of halofuginone.

Preferable dosage of the compositions of the present invention, the duration of the treatment, the administration regimes and the mode of application will depend on parameters associated with the phenomena to be treated as well as on characteristics of the treated subject (age, size, gender, and the general condition of the subject treated). According to certain embodiments, the total amount of halofuginone to be administered may be between about 0.005 and about 0.20 mg/Kg Body-Weight/day. The most common adverse events reported in connection with oral administration of a racemic mixture of halofuginone were nausea and vomiting (PCT Publication WO 2005/079703, the content of which is incorporated herein in full by reference). According to the disclosure of that PCT Application, these adverse effects may be overcome by a split-dose regimen of halofuginone. However, an administration regime of more than three times a day is highly non-desirable and would typically results and a very low patient compliance. Advantageously, the present invention now discloses pharmaceutical compositions comprising a highly pure D-enantiomer of halofuginone, which retain the biological activity of the racemic halofuginone mixture, while being devoid of its adverse effects.

According to certain embodiments, the compositions of the present invention are useful in the treatment of skin diseases and disorders including psoriasis, keloid, hypertrophic scar, acne, seborrhea and alopecia. Preferably, the skin disorder is selected from the group consisting of psoriasis, keloid and hypertrophic scar. Other indications for the use of the compositions of the present invention include fibrotic diseases such as scleroderma, graft versus host disease (GVHD), hepatic cirrhosis, pulmonary fibrosis, and renal fibrosis. Proliferative indications include restenosis, angiogenesis associated diseases and cancer, including malignant and non-malignant tumors. The present invention is meant to also encompass the use of the pharmaceutical compositions for induction of wound healing and prevention of adhesions.

In one embodiment of the present invention, the compounds of general formula (Ia) are useful for the preparation of a medicament for preventing and treating proliferative diseases or disorders including angiogenesis associated diseases, particularly tumor angiogenesis and tumor progression. In another embodiment of the present invention, the compounds are useful for preventing, treating or inhibiting a malignant cell proliferative disease or disorder. According to these two embodiments, the compounds are useful for the treatment or prevention of non-solid cancers, e.g. hematopoietic malignancies including, but not limited to, all types of leukemias and lymphomas, as well as of solid tumors such as, but not being limited to, mammary, ovarian, prostate, colon, cervical, gastric, esophageal, papillary thyroid, pancreatic, bladder, colorectal, melanoma, small-cell lung and non-small-cell lung cancers, granulosa cell carcinoma, transitional cell carcinoma, vascular tumors, all types of sarcomas, e.g. osteosarcoma, chondrosarcoma, Kaposi's sarcoma, myosarcoma, hemangiosarcoma, and glioblastomas.

It is to be understood that the terms “treating a disease or disorder associated with cell proliferative” or “treating a proliferative disease or disorder” encompass tumors, restenosis and fibrosis. Furthermore, the terms “treating or inhibiting a non-solid cancer” and “treating or inhibiting a tumor” are intended to encompass tumor formation, primary tumors, tumor progression or tumor metastasis.

### Modes of Administration

Oral administration

Pharmaceutical compositions for oral administration may be formulated as liquid or solid dosage form.

In some embodiments the pharmaceutical compositions for oral administration are formulated in a form selected from the group consisting of sterile solutions, sterile suspensions, sterile dry soluble lyophilized powders ready for reconstitution by combination with a vehicle just prior to use, sterile emulsions, microemulsions, dispersions, liposomal dosage forms, lipid complexes such as with cholesterol derivatives and phospholipids.

In some embodiments the solutions and vehicles are selected from aqueous and non-aqueous solutions. Preferably, aqueous vehicles for oral solutions are selected from the group consisting of sterile water and sodium chloride.

The oral formulation may be prepared as to further comprise an acid compound selected from the group consisting of glycolic, lactic, malic, maleic, citric, ascorbic and benzoic acid. In a preferred embodiment the aqueous oral solutions or vehicles further comprise buffering agents. According to one embodiment, the pH of the aqueous oral solution is in the range of about 3.5 to about 8.5. According to one currently preferred embodiment, the pH of the oral formulation is below 7.0, preferably below 6.5, more preferably below 6.0 and most preferably below 5.5

In other embodiments, the aqueous oral vehicle may further comprise cosolvents such as ethyl alcohol, polyethylene glycol, propylene glycol and mixtures thereof.

In one embodiment the sterile formulation may comprise lyophilized powders ready for reconstitution by aqueous vehicle. Such lyophilized powders comprise a D-enantiomer of quinazolinone derivative and a solid pharmaceutically acceptable buffering agent or a water-soluble organic acid. The buffering agents or organic acids used in the composition may be any non-toxic buffering agent or organic acid approved for oral use.

Optionally, at least one additional ingredient selected from the group consisting of preservatives, antioxidants and tonicity controlling agents may be added to the formulation. In one embodiment the preservatives are selected from the group consisting of benzyl alcohol, methyl paraben, propyl paraben, sodium salts of methyl paraben.

In another embodiment the tonicity controlling agents are selected from the group comprising of sodium chloride, mannitol, dextrose, glucose, lactose and sucrose.

In other embodiments the present invention relates to a solid pharmaceutical compositions for oral administration selected from the group consisting of tablets, capsules, sachets, powders, granules and lozenges.

In certain embodiments the present invention relates to a solid pharmaceutical composition formulated as tablets containing in addition to the active compound suitable excipients including, but not limited to, starches, gum arabic, calcium silicate, microcrystalline cellulose, polyvinylpyrrolidone, cellulose, water, syrup, and methylcellulose. The formulations can additionally include lubricating agents such as, for example, talc, magnesium stearate and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to: mannitol, sorbitol, xylitol, sucrose, maltose, glucose, lactose, dextrose, and the like. Suitable buffers encompass, but are not limited to, phosphate, citrate, tartarate, succinate, and the like. Other inert fillers, which may be used, encompass those which are known in the art and are useful in the manufacture of various dosage forms. If desired, the solid pharmaceutical compositions may include other components such as bulking agents and/or granulating agents, and the like. The compositions of the invention can be formulated so as to provide quick, sustained, or delayed release of the active ingredient after administration to the patient by employing procedures well known in the art.

In other embodiments the solid formulation may further comprise an acid compound selected from the group consisting of ascorbic, citric maleic and stearic acid.

Topical Application

The pharmaceutical compositions of the present invention formulated for topical use are selected from the group consisting of cream, ointment, lotion, gel, foam, suspension, aqueous or cosolvent solutions, salve, liposomes and any other pharmaceutically acceptable carrier suitable for topical administration of the drug.

In some embodiments the topical formulation is selected from the group consisting of emulsions, non-washable (water-in-oil) creams or washable (oil-in-water) creams, lotions, salves and the like.

As is well known in the art the physico-chemical characteristics of the carrier may be manipulated by addition of a variety of excipients, including but not limited to thickeners, gelling agents, wetting agents, flocculating agents, suspending agents and the like. These optional excipients will determine the physical characteristics of the resultant formulations such that the application may be more pleasant or convenient. It will be recognized by the skilled artisan that the excipients selected, should preferably enhance and in any case must not interfere with the storage stability of the formulations.

The halofuginone concentration in the topical compositions is in the range 0.0001-10% (w/w) and most preferably in the range of 0.001-2% (w/w).

In certain embodiments the pharmaceutical composition for use in the topical formulations of the invention further comprises an acid compound selected from the group consisting aliphatic acids, aromatic, acetic, ascorbic or benzoic acid, citric, glycolic, lactic, malic and maleic acid. According to one currently preferred embodiment, the acid compound for use in the topical formulation of the invention is lactic acid.

According to one currently preferred embodiment the present invention provides a cream formulation comprising in addition to the active compound: (a) a hydrophobic component; (b) a hydrophilic aqueous component; and (c) at least one emulsifying agent, wherein the pH of the aqueous component is less than 7.0.

Preferably the hydrophobic component of the cream is present in an amount from about 10% to about 90% (w/w) based on the total weight of the composition and most preferably the hydrophobic component of the cream is present in an amount from about 20% to about 80% (w/w) based on the total weight of the composition.

The hydrophobic component of the cream is exemplified by the group consisting of mineral oil, yellow soft paraffin (Vaseline), white soft paraffin (Vaseline), paraffin (hard paraffin), paraffin oil heavy, hydrous wool fat (hydrous lanolin), wool fat (lanolin), wool alcohol (lanolin alcohol), petrolatum and lanolin alcohols, beeswax, cetyl alcohol, almond oil, arachis oil, castor oil, hydrogenated castor oil wax, cottonseed oil, ethyl oleate, olive oil, sesame oil, and mixtures thereof.

Water alone, propylene glycol or alternatively any pharmaceutically acceptable buffer or solution exemplifies the hydrophilic aqueous component of the cream. Preferably, the hydrophilic aqueous component of the cream is present in an amount from about 10% to about 90% (w/w) based on the total weight of the composition and most preferably the hydrophilic aqueous component of the cream is present in an amount from about 20% to about 80% (w/w) based on the total weight of the composition.

Exemplary buffers are acetate, borate (borax), citrate, lactate, phosphate and mixtures thereof.

Emulsifying agents may be added to the cream in order to stabilize the cream and to prevent the coalescence of the droplets. The emulsifying agent reduces the surface tension and forms a stable, coherent interfacial film.

Suitable hydrophilic emulsifying agents comprising the complex emulgator are known in the art and may be exemplified but not limited to the group consisting of polyoxyethylene sorbitan monolaurate (Tween® 20), polyoxyethylene sorbitan monopalmitate (Tween® 40), polyoxyethylene sorbitan monostearate (Tween® 60), polyoxyethylene sorbitan monooleate (Tween® 80), polyoxyethylene lauryl ether (Brij 35), polyoxyethylene castor oil (Atlas G-1794), sodium lauryl sulfate, cetrimide, cetomacrogol and mixtures thereof.

Suitable hydrophobic emulsifying agents comprising the complex emulgator are known in the art and may be exemplified but not limited to the group consisting of sorbitan trioleate (Span 85, Aracel 85), sorbitan tristearate, sorbitan sesquioleate (Aracel 83), (Span 65), sorbitan monooleate (Span 80), propylene glycol monostearate, sorbitan sequioleate (Aracel C), glycerol monostearate, propylene glycol monolaurate (Atlas G-917, Atlas G-3851), sorbitan monostearate (Span 60, Aracel 60), sorbitan monopalmitate (Span 40, Aracel 40), sorbitan monolaurate (Span 20, Aracel 20), cetostearyl alcohol, cetyl alcohol, oleic acid, stearic acid and mixtures thereof.

A suitable emulsifying agent may be exemplified by but not limited to the group consisting of cholesterol, cetostearyl alcohol, wool fat (lanolin), wool alcohol (lanolin alcohol), hydrous wool fat (hydrous lanolin), and mixtures thereof.

In one embodiment the concentration of the at least one emulsifying agent in the cream is in the range from about 2% to about 40% (w/w) based on the total weight of the composition.

In another embodiment pharmaceutical composition of the present invention is formulated in the form of aqueous suspensions. In a preferred embodiment the suspension comprising in addition to the active compound: (a) an aqueous medium; and (b) suspending agents or thickeners. (c) an acid compound, optionally additional excipients are added, as specified heretofore.

Suitable suspending agent or thickeners may be exemplified by but not limited to the group consisting of cellulose derivatives like methylcellulose, hydroxyethylcellulose and hydroxypropyl cellulose, alginic acid and its derivatives, xanthan gum, guar gum, gum arabic, tragacanth, gelatin, acacia, bentonite, starch, microcrystalline cellulose, povidone and mixtures thereof.

In some embodiments the suspending agents or thickeners are present in an amount from about 0.1% to about 15% (w/w) based on the total weight of the composition.

In another embodiment the aqueous suspensions may optionally contain additional excipients selected from the group consisting of wetting agents, flocculating agents, thickeners, and the like.

Suitable wetting agents are exemplified by but not limited to the group consisting of glycerol polyethylene glycol, polypropylene glycol and mixtures thereof, and surfactants. The concentration of the wetting agents in the suspension should be selected to achieve optimum dispersion of the pharmaceutical powders within the suspension with the lowest feasible concentration of the wetting agent.

Suitable flocculating agents are exemplified by but not limited to the group consisting of electrolytes, surfactants, and polymers.

The suspending agents, wetting agents and flocculating agents are provided in amounts that are effective to form a stable suspension of the pharmaceutically effective agent.

In another preferred embodiment the gel formulation of the present invention comprises in addition to the active compound, at least one gelling agent and an acid compound.

Suitable gelling agents may be exemplified by but not limited to the group consisting of hydrophilic polymers, natural and synthetic gums, crosslinked proteins and mixtures thereof. In a preferred embodiment the polymers are selected from the group consisting of hydroxyethylcellulose, hydroxyethyl methylcellulose, methyl cellulose, hydroxypropylcellulose, hydroxypropyl methylcellulose, carboxymethyl cellulose, and similar derivatives of amylose, dextran, chitosan, pullulan, and other polysaccharides; Crosslinked proteins such as albumin, gelatin and collagen; acrylic based polymer gels such as Carbopol, Eudragit and hydroxyethyl methacrylate based gel polymers, polyurethane based gels and mixtures thereof.

In some embodiments the gums are selected from the group consisting of acacia, agar, carageenan, dextrin, gelatin, guar gum, hyaluronic acid, tragacanth gum, xanthan gum, and mixtures thereof. In a preferred embodiment the gelling agent is present in an amount from about 1% to about 25% (w/w) based on the total weight of the composition. In a preferred embodiment the pH of the aqueous phase of the gel is in the range 1.0-6.8.

In another embodiment pharmaceutical compositions of the present invention are formulated as a solution. Such a solution comprises, in addition to the active compound, an acid compound and at least one co-solvent exemplified but not limited to the group consisting of water, buffered solutions, organic solvents such as ethyl alcohol, isopropyl alcohol, propylene glycol, polyethylene glycol, glycerin, glycoforol, Cremophor®, ethyl lactate, methyl lactate, N-methylpyrrolidone, ethoxylated tocopherol and mixtures thereof.

In a preferred embodiment the solution comprises a mixture of the active agent dissolved or dispersed in an aqueous solution of a pH range between 1 and 6.8. Alternatively cosolvent solutions of halofuginone may be prepared using pharmaceutically acceptable organic solvents such as ethanol, isopropanol, glycerol, propylene glycol, low molecular weight poly(ethylene glycol) and its copolymers with propylene glycol. The solutions may be maintained as a mixture of hydrophilic components or contain water at various amounts for oral and systemic as well as topical use.

The preferred concentration of halofuginone in solutions is between about 0.0001% to about 1% (w/w). According to currently certain preferred embodiments, the solutions containing water should be kept at a pH range between 1 and 6.8 to avoid irritation or degradation of the drug, halofuginone.

The topical composition of the present invention may optionally contain at least one additional ingredient, selected from the group consisting of preservatives, antioxidants, humectants, emollients, thickeners, structuring agents, stabilizers, coloring agents, and perfumes.

The creams, ointments, lotions and gels may be prepared by incorporating halofuginone in a finely-divided or powdered form alone or in solution or suspension, in an aqueous or non-aqueous fluid, to the pharmaceutical carrier.

Halofuginone may be dissolved, dispersed, suspended or partially dispersed and partially dissolved in the pharmaceutical carrier, depending on the solubility of halofuginone in the selected pharmaceutical carrier.

Parenteral Administration

Pharmaceutical compositions for parenteral administration are formulated for intravenous injections, intravenous infusion, intradermal, intralesional, intramuscular, and subcutaneous injections or depots; or they may be administered parenterally by means other than an injection, for example, it could be introduced laparascopically, intravesicularly, or via any orifice not related to the gastrointestinal tract.

In one embodiment the pharmaceutical compositions for parenteral administration are preferably a formulation selected from the group consisting of sterile solutions ready for injection, sterile suspensions ready for injection, sterile dry soluble lyophilized powders ready for reconstitution by combination with a vehicle just prior to use, sterile emulsions, microemulsions, dispersions, liposomal dosage forms, lipid complexes such as with cholesterol derivatives and phospholipids.

In a preferred embodiment the solutions and vehicles are selected from the group consisting of aqueous or non-aqueous solutions. In a preferred embodiment the aqueous parenteral solutions and vehicles are selected from the group consisting of sterile water for injection, sodium chloride injection, Ringer's injection, Isotonic Dextrose injection, Dextrose and Lactated Ringer's injection.

In some embodiments the aqueous parenteral solutions or vehicles further comprise buffering agents. In a currently preferred embodiment, the pH of the aqueous parenteral solution is in the range of 3.5 to 6.0. The acid compound, for the parenteral formulations, is selected from the group consisting of ascorbic and benzoic acid, citric glycolic, lactic, malic and maleic acid.

The aqueous parenteral vehicle may further comprise cosolvents also referred to as water miscible solvents such as ethyl alcohol, polyethylene glycol, propylene glycol and mixtures thereof.

The sterile injection may comprise lyophilized powders ready for reconstitution by aqueous vehicle. Such lyophilized powders comprising quinazolinone derivative and a solid pharmaceutically acceptable buffering agent or a water-soluble organic acid. The buffering agents or organic acids used in the composition may be any non-toxic buffering agent or organic acid approved for parenteral use.

In a currently preferred embodiment the buffering agent or organic acid are present in amount such that the pH of the formulation upon reconstitution with water or other pharmaceutically acceptable vehicle is between about 3.5 to about 6.0.

Optionally, at least one additional ingredient selected from the group consisting of preservatives, antioxidants and tonicity controlling agents can be used.

Preservatives may be selected from the group consisting of benzyl alcohol, methyl paraben, propyl paraben, and sodium salts of methyl paraben. Tonicity controlling agents are selected from the group consisting of sodium chloride, mannitol, dextrose, glucose, lactose and sucrose.

It is to be understood that the invention is not limited in its application to the details of construction and arrangement of the components set forth in the following description. The invention includes other embodiments and can be practiced or implemented in various ways. Also it is to be understood that the phraseology and terminology employed herein is for the purpose of description only and should not be regarded as limiting.

### EXAMPLES

### Example 1

### Purification of The Halofuginone Enantiomers

The D- and L-enantiomers of halofuginone were isolated from a racemic mixture on a Chirobiotic V2 column (250×21.2 mm, 5 u silica) of Advanced Separation Technologies Inc. Four milligrams of racemic material was loaded onto the column. The mobile phase was a 90/10, 0.2w % NH₄TFA in MeOH/H₂O. The flow rate was set to 16 ml/min. The UV absorption was set to 243 nm, temperature of 23° C. This procedure was repeated as necessary to obtain a sufficient quality and quantity of the desired enantiomer.

The retention time for the D-enantiomer (enantiomer 1) was 14.2 minutes. Chiral purity was determined to be 99.80%. The retention time for the L-enantiomer (enantiomer 2) was 16.2 minutes. Chiral purity was determined to be 98.97%. The isolated enantiomers were further analyzed in in vitro and in vivo assays. Purity was determined by analytical chiral HPLC.

### Example 2

### Inhibition of Cell Proliferation by The D-Enantiomer

The present study sought to compare the effect of each of the trans-halofuginone enantiomers to the racemic mixture on proliferation of cultured, actively growing cells.

Cells

Human aortic smooth muscle cells (hAoSMC) and Human Umbilical Vein Endothelial Cells (HUVEC) were purchased from Clonetics and grown in Clonetics suitable growth media. Human skin fibroblasts (Detroit 551) and human fibrosarcoma (HT1080) cell lines were purchased from the American Type Culture Collection (ATCC) and grown in MEM supplemented with 10% FCS, 1 mM Sodium pyruvate, 2 mM L-glutamine and 0.2% antibiotic solution (Biological industries, Beit-Haemek, Israel). The human bladder carcinoma (5637) and human breast carcinoma (MDA-MB-435S) cell lines were purchased from the ATCC and grown in DMEM supplemented with 10% FCS and 0.2% antibiotic solution (Biological industries, Beit-Haemek, Israel). Cells were grown in 37° C. and 5% CO₂.

Cell Proliferation Assay

Cells were seeded in 96-well plates. Twenty-four hours later, increasing concentrations of tested compounds were added to the wells. When the cultures reached sub confluence (72-96 hours after seeding) the cells were fixed with 2.5% glutaraldehyde, washed three times with distilled water, followed by one wash with 0.1 M Boric buffer (pH 8.5). Thereafter, 100 μL Methylene blue (1% in Boric buffer, pH 8.5) were added to each well and incubated for 60 min at room temperature. Cells were washed extensively in distilled water in order to remove non-cell-bound dye. The plates were then dried and the methylene blue was extracted with 200 μL/well of 0.1 N HCl for 60 min at 370 C. The optical density at 620 nm was determined using a micro plate spectrophotometer.

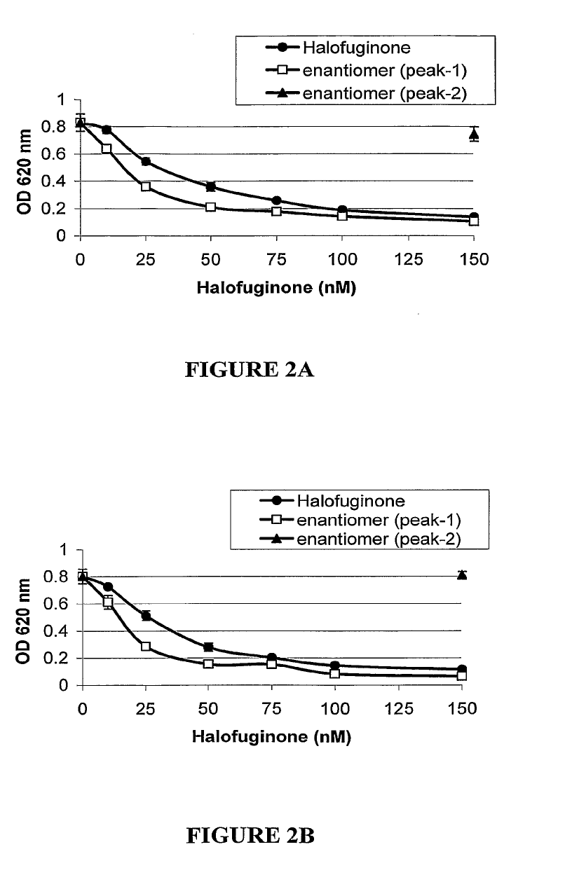

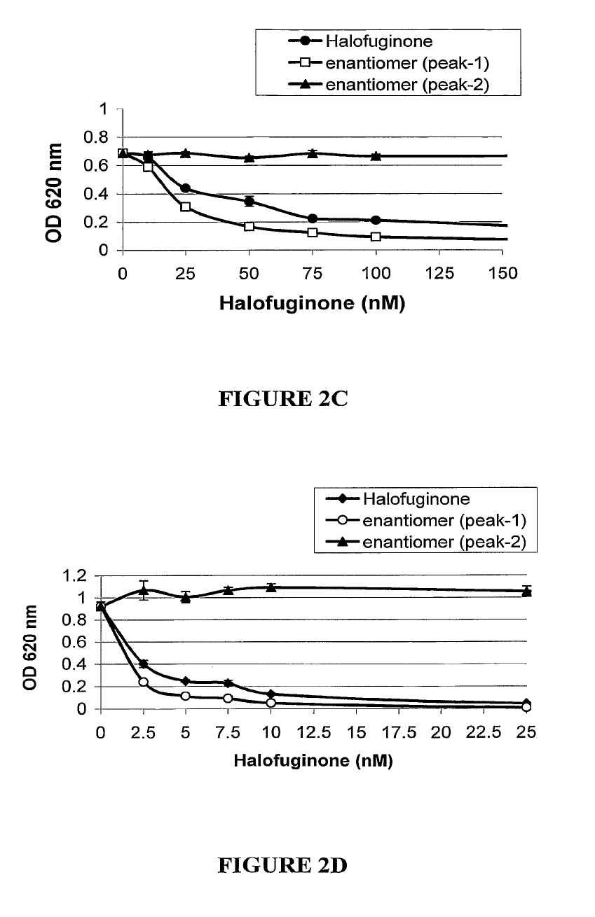

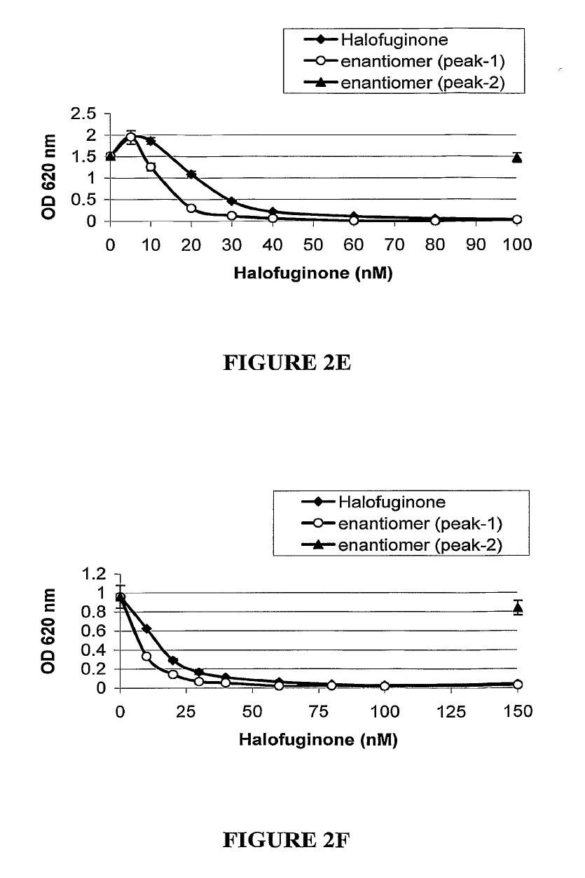

FIG. 2 shows growth curves of different cell lines treated with the purified D-enantiomer, the purified L-enantiomer obtained as described in Example 1 herein above, or the racemic mixture of halofuginone. The D-enantiomer (enantiomer 1) suppressed proliferation of all the tested cells in a dose dependent manner and it was about two fold more active then the racemic mixture. In contrast, the L-enantiomer (enantiomer 2) had no effect on cell proliferation in all tested cells.

### Example 3

### Inhibition of Collagen Synthesis of Fibroblasts The D-Enantiomer

Progressive fibroproliferative diseases such as liver cirrhosis, pulmonary and kidney fibrosis, scleroderma, etc., exhibit excessive production of connective tissues, which results in destruction of normal tissue architecture and function. The fibrotic reaction is thought to involve the stimulative response of tissue cells resulting in increased proliferation as well as extracellular matrix (ECM) deposition. Collagen was found to be a major ECM molecule synthesized in the fibrotic lesion. In some cases, such as in pulmonary and kidney fibrosis, the fibroblasts are thought to play a pivotal role.

Skin fibroblasts (Detroit 551) were seeded in a 96-well plate (3000 cells/well). Twenty-four hours later the cells were incubated for additional 48 h with 75 μM ascorbic acid and increasing concentrations of the tested compounds (induction medium was exchanged after 24 h). Cell medium was collected and the Prolagen-C ELISA kit of “METRA” was used for detection of collagen type I C′ propeptide (CICP) concentration.

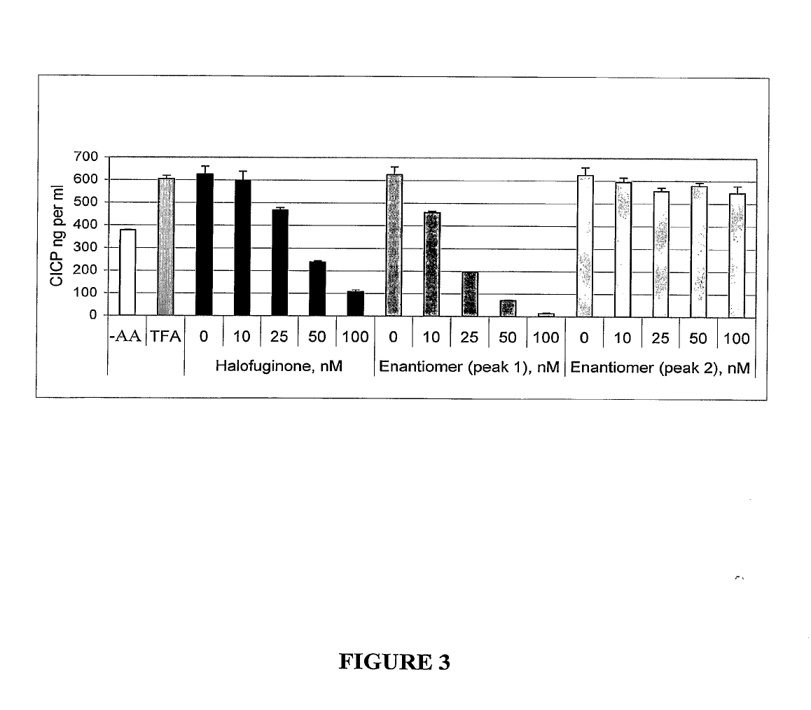

FIG. 3 shows that the D-enantiomer of halofuginone (enantiomer 1) suppressed collagen type I synthesis of skin fibroblasts in a dose dependent manner and it was about two fold more active then the racemic mixture. In contrast, the L-enantiomer (enantiomer 2) had no effect on collagen synthesis of fibroblasts.

### Example 4

### Toxicity Study of The Halofuginone D-Enantiomer

The object of the study is to obtain the No Effect Level and toxic dose values of pure D-enantiomer halofuginone in a 4-week study by oral gavage, in canines.

Three groups of purebred beagle dogs receive the purified D-enantiomer halofuginone by once daily oral gavage administration at dosages of 0.0375, 0.07 or 0.15 mg/kg/day for 4 weeks. A fourth group receives the vehicle alone at the same frequency and serves as control.

Clinical signs, body weight and food consumption data are recorded throughout the study. Sperm analysis, electrocardiology, blood pressure measurements, ophthalmic examinations and laboratory investigations (hematology, blood chemistry, urinalysis, and fecal occult blood) are conducted at intervals. At the end of the study, all surviving dogs are humanely killed, bone marrow smears are taken for macroscopic examination and selected organ weights are recorded. Body tissues are preserved for subsequent histopathological examination.

The results include recordation of unscheduled deaths and the effect of treatment on electrocardiography, blood pressure, sperm count and viability, urinalysis and blood chemistry. Ophthalmic checkups are included.

Clinical signs including loose/liquid feces, vomiting, salivation, body weight, quiet behavior and body temperature are recorded.

### Example 5

### Preparation of Halofuginone D-Enantiomer Formulations

Preparation of Halofuginone Solution for Gavage

400 μl lactic acid is dissolved in 5 ml of double distilled (DD) water. The pH of the mixture is adjusted to pH 4.0-4.5 with ˜500 μl NaOH 30% (w/v in DD water). The volume of the solution is brought to 10 ml with DD water. The isolated D-enantiomer of halofuginone obtained as described in Example 1 herein above is added to a final concentration of 1 mg/ml (5 mg halofuginone in 5 ml of the above described solution). The solution is then heated at 40-45° C., for less than 1 minute until full dissolution. The resulting stock solution may be diluted with DD water to any required concentration before injection.

Preparation of Halofuginone Solution for Injection

400 μl lactic acid is dissolved in 5 ml of double distilled (DD) water. The pH of the mixture is adjusted to pH 4.0-4.5 with ˜500 μl NaOH 30% (w/v in DD water). The total volume of the solution is brought to 10 ml with DD water. The isolated D-enantiomer of halofuginone obtained as described in Example 1 herein above is added to a final concentration of 1 mg/ml (5 mg halofuginone in 5 ml of the above described solution). The solution is then heated at 40-45° C., for less than 1 minute until full dissolution. The resulting stock solution is diluted with saline to any required concentration below 0.25 mg/ml and then filter-sterilized through 0.2μ filter.

Topical Application

Formulations for topical application can include gels, suspensions, aqueous or cosolvent solutions, salves, liposomes and creams.

Preliminary formulations for topical application of isolated D-enantiomeric halofuginone may be prepared in two ways: hydrophilic gel and lipophilic cream.

Hydrophilic gel: The gel is prepared with a jellifying substance: polyethyleneglycol (Poloxamer-Lutrol F 127) or a synthetic cellulose derivative—Hydroxyethylcellulose—(Natrosol 250 HX Pharm).

All the preparations are physically stable at +40° C.

Lipophilic cream: A water in oil (w/o) cream containing heavy paraffin oil may be prepared using a silicone polymer emulsifier (cetyl dimethicone copolyol and polyglyceryl-4-isostearate, Abil WE09). The advantage of this system is that no heating is required during the process and the emulsion is very stable.

Variants of the above formulation, with various consistencies (viscosity), are prepared. Silicone polymer is widely used as emulsifier in cosmetics, however it is not registered in any Pharmacopoeia. Therefore, the emulsifier may be replaced by sorbitan sesquioleate (Arlacel 83), a nonionic emulgator specific for emulsion w/o. The oily phase containing paraffin oil, and a synthetic wax: hydrogenated castor oil (castor wax) may be used as a thickener. The stability of the prototypes is measured under various conditions.

Formulation for Oral Administration

In the formulation of a tablet dosage form for oral administration the active ingredient is applied in its dried form. Tablets were prepared comprising 0.5 mg halofuginone, 4 mg citric acid, 2 mg Mg Stearate NF, 4 mg talc USP, 89.3 mg Microcrystaline Cellulose NF, 4 mg Crospovidine NF, 0.2 mg Colloidal Silicon Dioxide NF, and 96 mg Lactose Monohydrate NF.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A pharmaceutical composition for the treatment of diseases and disorders associated with fibrotic conditions or cell proliferation, the composition comprising as an active ingredient an isolated quinazolinone derivative having the general formula (Ia): 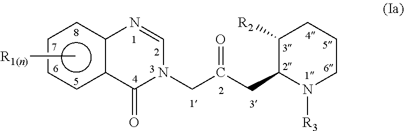
having the (2″R,3″S)trans configuration and being essentially free of the (2″S,3″R)trans enantiomer, wherein:
n=1-2,
R₁ is at each occurrence independently selected from the group consisting of hydrogen, halogen, nitro, lower alkyl, phenyl and lower alkoxy;
R₂ is a member of the group consisting of hydroxy, acetoxy and lower alkoxy;
and R₃ is a member of the group consisting of hydrogen and lower alkenoxy-carbonyl, and pharmaceutically acceptable salts thereof, further comprising a pharmaceutically acceptable carrier or diluent, the quinazolinone derivative being at least 95% chirally pure (2″R,3″S)trans enantiomer.

2. The pharmaceutical composition according to claim 1 wherein the composition comprises at least 98% chirally pure (2″R,3″S)trans enantiomer.

3. The pharmaceutical composition according to claim 1 wherein the composition comprises at least 99% chirally pure (2″R,3″S)trans enantiomer.

4. The pharmaceutical composition according to claim 1 wherein the active ingredient is an isolated (2″R,3″S)trans enantiomer of halofuginone or a pharmaceutically acceptable salt of halofuginone.

5. The pharmaceutical composition according to claim 1 wherein the pH of the composition is from about 3.5 to about 8.5.

6. The pharmaceutical composition according to claim 5, wherein the pH of the composition is below 7.0.

7. The pharmaceutical composition according to claim 6, wherein the pH of the composition is below 6.0

8. The pharmaceutical composition according to claim 7, wherein the pH of the composition is below 5.5

9. The pharmaceutical composition according to claim 4, wherein the concentration of halofuginone is in the range of about 0.0001% to about 30% (w/w).

10. The pharmaceutical composition according to claim 9 wherein the concentration of halofuginone is in the range of about 0.001% to about 2%.

11. The pharmaceutical composition according to claim 1, wherein the composition exhibits reduced side effects obtained upon administering a pharmaceutical composition comprising a racemic mixture of the quinazolinone derivative.

12. The pharmaceutical composition according to claim 11, wherein the reduced side effects include at least one reduced adverse effects selected from nausea, vomiting, salivation, diarrhea, apathy, low sperm count, vision problems, hypertension, hypotension, hypothermia and deviation from normal hematological counts.

13. The pharmaceutical composition according to claim 1 for topical, parenteral or oral administration.

14. The pharmaceutical composition according to claim 13 wherein the topical form is selected from the group consisting of cream, ointment, lotion, gel, suspension, aqueous or cosolvent solution, salve and liposomes.

15. The pharmaceutical composition according to claim 13 formulated for parenteral administration selected from a group consisting of forms suitable for intravenous injections, intravenous infusion, intradermal, intralesional, intramuscular and subcutaneous injections or depots, or for administering laparascopically and intravesicularly.

16. The pharmaceutical composition according to claim 15 wherein the pharmaceutical composition is selected from the group consisting of sterile solutions ready for injection, sterile suspensions ready for injection, sterile dry soluble lyophilized powders ready for reconstitution by combination with a vehicle just prior to use, sterile emulsions, microemulsions, dispersions, liposomal dosage forms and lipid complexes.

17. The pharmaceutical composition according to claim 13 wherein the oral form is a solid formulation selected from the group consisting of tablets, capsules, sachets, powders, granules and lozenges.

18. The pharmaceutical composition according to claim 13 wherein the oral form is a liquid formulation selected from the group consisting of an aqueous formulation and a non-aqueous formulation.

19. The pharmaceutical composition according to claim 1 wherein the diseases and disorders associated with fibrotic conditions or cell proliferation are selected from the group consisting of psoriasis, keloid, hypertrophic scar, acne, seborrhea, alopecia, scleroderma, graft versus host disease (GVHD), hepatic cirrhosis, pulmonary fibrosis, renal fibrosis, restenosis and malignant and non-malignant tumors.

20. The pharmaceutical composition according to claim 4 wherein the diseases and disorders associated with fibrotic conditions or cell proliferation are selected from the group consisting of psoriasis, keloid, hypertrophic scar, acne, seborrhea, alopecia, scleroderma, graft versus host disease (GVHD), hepatic cirrhosis, pulmonary fibrosis, renal fibrosis, restenosis and malignant and non-malignant tumors.

21. A method of treating a disease or disorder associated with fibrotic conditions or cell proliferation, comprising administering to a subject in need thereof a pharmaceutical composition comprising as an active ingredient a therapeutically effective amount of an isolated quinazolinone derivative having the general formula (Ia): 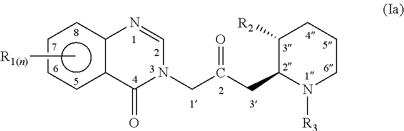
having the (2″R,3″S)trans configuration and being essentially free of the (2″S,3″R)trans enantiomer, wherein:
n=1-2,
R₁ is at each occurrence independently selected from the group consisting of hydrogen, halogen, nitro, lower alkyl, phenyl and lower alkoxy;
R₂ is a member of the group consisting of hydroxy, acetoxy and lower alkoxy;
and R₃ is a member of the group consisting of hydrogen and lower alkenoxy-carbonyl, and pharmaceutically acceptable salts thereof, and a pharmaceutically acceptable carrier or diluent, the composition comprising at least 95% chirally pure (2″R,3″S)trans enantiomer of the quinazolinone derivative.

22. The method according to claim 21 wherein the composition comprises at least 98% chirally pure (2″R,3″S)trans enantiomer.

23. The method according to claim 21 wherein the composition comprises at least 99% chirally pure (2″R,3″S)trans enantiomer.

24. The method according to claim 21 wherein the active ingredient is an isolated (2″R,3″S)trans enantiomer of halofuginone or a pharmaceutically acceptable salt of halofuginone.

25. The method according to claim 21 wherein the pH of the composition is from about 3.5 to about 8.5.

26. The method according to claim 25, wherein the pH of the composition is below 7.0.

27. The method according to claim 26, wherein the pH of the composition is below 6.0

28. The method according to claim 27, wherein the pH of the composition is below 5.5

29. The method according to claim 24, wherein the concentration of halofuginone is in the range of about 0.0001 to about 30%.

30. The method according to claim 29 wherein the concentration of halofuginone is in the range of about 0.001% to about 2%.

31. The method according to claim 21, wherein administering the pharmaceutical composition results in reduced side effects compared to the side effects obtained upon administering a pharmaceutical composition comprising a racemic mixture of the quinazolinone derivative.

32. The method according to claim 31, wherein the reduced side effects include at least one reduced adverse effect selected from nausea, vomiting, salivation, diarrhea, apathy, low sperm count, vision problems, hypertension, hypotension, hypothermia and deviation from normal hematological counts.

33. The method according to claim 21 wherein the composition is selected from a topical formulation, a parenteral formulation and an oral formulation.

34. The method according to claim 33 wherein the topical form is selected from the group consisting of cream, ointment, lotion, gel, suspension, aqueous or cosolvent solution, salve and liposomes.

35. The method according to claim 33 formulated for parenteral administration selected from a group consisting of forms suitable for intravenous injections, intravenous infusion, intradermal, intralesional, intramuscular and subcutaneous injections or depots, or for administering laparascopically and intravesicularly.

36. The method according to claim 35 wherein the pharmaceutical composition is selected from the group consisting of sterile solutions ready for injection, sterile suspensions ready for injection, sterile dry soluble lyophilized powders ready for reconstitution by combination with a vehicle just prior to use, sterile emulsions, microemulsions, dispersions, liposomal dosage forms and lipid complexes.

37. The method according to claim 33 wherein the oral form is a solid formulation selected from the group consisting of tablets, capsules, sachets, powders, granules and lozenges.

38. The method according to claim 33 wherein the oral form is a liquid formulation selected from the group consisting of an aqueous formulation and a non-aqueous formulation.

39. The method according to claim 21 wherein the diseases and disorders associated with fibrotic conditions or cell proliferation are selected from psoriasis, keloid, hypertrophic scar, acne, seborrhea, alopecia, scleroderma, graft versus host disease (GVHD), hepatic cirrhosis, pulmonary fibrosis, renal fibrosis. restenosis and malignant and non-malignant tumors.

40. The method according to claim 24 wherein the diseases and disorders associated with fibrotic conditions or cell proliferation are selected from psoriasis, keloid, hypertrophic scar, acne, seborrhea, alopecia, scleroderma, graft versus host disease (GVHD), hepatic cirrhosis, pulmonary fibrosis, renal fibrosis. restenosis and malignant and non-malignant tumors.

41. The method of claim 21 wherein the subject is a human subject.
